(19) United States
(12) Patent Application Publication
(10) Pub. No.: US 2021/0371941 A1
(43) Pub. Date: Dec. 2, 2021

(54) SYSTEMS AND METHODS FOR DETECTION OF LOW-COPY NUMBER NUCLEIC ACIDS

(71) Applicant: Pero Dimsoski, Brentwood, TN (US)

(72) Inventor: Pero Dimsoski, Brentwood, TN (US)

(21) Appl. No.: 16/890,596

(22) Filed: Jun. 2, 2020

(51) Int. Cl. C12Q 1/70

(52) U.S. Cl. CPC C12Q 1/701

(57) ABSTRACT

Disclosed are compositions, methods, and systems for genetic identification and detection of low-copy number nucleic acids, and provides method, compositions, and kits useful for this purpose. The methods of the invention can be used to detect in a given sample the presence of low-copy number of nucleic acids (e.g., DNA or RNA) of various microorganisms, including SARS-COV-2.

### FIELD OF THE INVENTION

The present invention relates generally to the field of genetic identification and detection of low-copy number nucleic acids, and provides systems, methods, compositions, and kits useful for this purpose. The systems, compositions, and methods of the invention can be used to detect in a given sample the presence of nucleic acids (e.g., DNA and/or RNA) of various microorganisms, including SARS-COV-2.

### BACKGROUND

The determination of nucleic acids such as the DNA or RNA genotype at a given locus, or loci, or sequence of DNA region, or whole genomic DNA or RNA, can be achieved by polymerase chain reaction (PCR). The PCR-based methods are useful when the DNA quantity is low or limited, therefore, it is generally accepted to describe the PCR methods as relatively sensitive in amplifying low quantity of DNA or RNA. In addition, it is generally accepted, that the PCR-based methods for amplification of DNA or RNA, due to low variation of primary structure of some DNA or RNA regions of the genome of particular organism, also known as conserved DNA/RNA regions, make possible to design a complementary-to-conserved regions DNA sequences, also known as primers, that are starting point of amplification of the targeted DNA region, that would hybridize to conserved regions of the DNA or RNA at a specific Tm (melting temperature), thus, contributing to the high specificity of the PCR assay.

The standard PCR method can be efficiently used with one nanogram to one microgram of input DNA or RNA. However, often in practice, there is a need to amplify, genotype or sequence DNA or RNA that is bellow one nanogram in quantity, or amplify, genotype or sequence a low-copy number DNA or RNA. This is the case when amplifying, genotyping or sequencing microbial genetic material such as a viral DNA or RNA, for example, the SARS-COV-2, Anthrax bacteria, or other microorganisms, particularly at the onset of infection, when the quantity of the microorganisms or infecting agents in the collected sample is presumably very low. During the PCR amplification and genotyping or sequencing of viral genetic material such as SARS-COV-2, or Anthrax bacteria, or other microorganisms that cause infectious diseases, in addition to specificity of the PCR reaction, which is the feature of the PCR reaction to amplify the DNA or RNA of the particular targeted microorganism like SARS-COV-2, Anthrax, or other microorganisms, also very important is the sensitivity of the detection method. This is because the more sensitive the method of detection is, the earlier the infection-causing microorganism can be identified or detected, and infection can be efficiently managed either by medical or epidemiological means or both.

The PCR method was originally invented to address the issue of detection of DNA material by amplifying or making large number of copies of targeted DNA fragments, which was the first step of detection process. This was followed by the second and final step of genotyping or sequencing of the PCR-amplified DNA on an instrument-platform. However, the PCR method is limited because it is yielding compounds, such as unincorporated primers, dNTPs, enzyme, buffer ions, etc., that are not only unnecessary for the detection process, but can imped the performance of the genotyping or sequencing platform.

In summary, the problem of early detection of the infectious microorganism is a problem of amplification of low-copy number DNA or RNA, therefore, the sensitivity of the detection method is the most important factor contributing toward early detection, genotyping or sequencing.

Sensitivity of the PCR assay is very important during amplification, genotyping or sequencing of circulating free DNA (cfDNA) and circulating tumor DNA (ctDNA) that are degraded, low-copy number DNA circulating in bloodstream and other body fluids and are reliable markers of cancer, or tumor DNA. It has been shown that the cfDNA and ctDNA can be used for diagnosis of prostate cancer, breast cancer, colorectal cancer, or other cancers and tumors. The cfDNA and ctDNA fragments are either short, between 70-200 bp in length, or long, over 20 kb in length. Thus, makes them particularly suitable targets for PCR amplification. The detection of the cfDNA and ctDNA in the bloodstream of the patient would help diagnose the status of patient's health with regard to the cancer or tumor's stage or detect free floating tumor DNA in bloodstream and other bodily fluids after surgery and tumor removal, and point toward the future treatment. For example, in one scenario, after removal of a tumor tissue, the most important information is to determine if any tumor cells are leftover in patient's body. If it is determined that there is no cfDNA or ctDNA associated with the particular tumor, that would mean that the patient is cancer/tumor free and would not be necessary to expose him/her to customary, but rather difficult, chemo or radiation treatment after surgery. In another scenario, early detection of cfDNA or ctDNA can lead to early diagnosis of cancer/tumor tissue at the onset of the diseases, and may lead to timely treatment and successful outcome. At issue here is also the detection of low quantity of cfDNA or ctDNA, therefore, the sensitivity of the detection method is the most important criteria for successful early diagnosis.

Also, during the library enrichment preparation of DNA or RNA for Sanger sequencing or Next Generation Sequencing (NGS), the goal is to obtain enough quantity of high quality (purified) DNA (or RNA), that is usually in single-strand configuration, or region(s) of DNA or RNA of interest that usually are in single-strand configuration, that is optimal input for the NGS instrument. The current methods for DNA or RNA library preparations for NGS or Sangers sequencing can require large quantities of genomic DNA or RNA that are fragmented, where target DNA is selected by specific DNA probes. In addition, the current methods may consist of multiple laboratory steps and lengthy workflow. Also, with some of the library preparation and enrichment methods of DNA samples there is an inherent loss of specificity and sensitivity due to use of specific probes in obtaining targeted DNA which altogether are limiting factors for Sanger sequencing or NGS when working with the low-copy number DNA.

The amplification, genotyping or sequencing of low-copy number DNA or RNA, such as the viral RNA or DNA obtained from samples at the onset of infection, or DNA library preparation of the low-copy number or degraded DNA for Sangers sequencing or NGS sequencing, such as the DNA or RNA extracted from the Formalin Fixed Paraffin Embedded (FFPE) samples, or cfDNA or ctDNA samples, is the same issue as amplifying, genotyping or sequencing, degraded DNA or RNA or other challenging samples. This is because the effect of degradation is to lower the number of amplifiable copies of DNA due to action of the restriction enzymes randomly shearing the DNA during the cell degradation, therefore, the low-copy number DNA and the degraded DNA are representing the same problem. The degraded DNA or RNA for all practical purposes of amplification, genotyping or sequencing, can be considered to be a low-copy number DNA or RNA. Several strategies can be utilized to amplify low-copy number DNA or RNA. Designing PCR amplification primers to hybridize close to the targeted regions of the DNA or RNA is one solution. This approach can help amplify low molecular weight DNA where targeted DNA is below 200 bp, which is less likely to be degraded, and improvement in sensitivity of detection can be up to 10% when compared to PCR-assay designed with primers to hybridize further away from the targeted sequence with expected PCR product size of >200 bp. Also, improving the extraction efficiency can facilitate the amplification of the low-copy number DNA because more efficient extraction will yield higher quantities of amplifiable DNA. Therefore, applying efficient DNA or RNA extraction method can also improve the sensitivity of detection by 5-7%. The post-PCR product purification can additionally improve the sensitivity of DNA or RNA detection. In addition, instrument improvement in detecting fluorescently labeled DNA fragments or nucleotides also can increase the sensitivity of detection.

Despite the desire to achieve higher sensitivity levels, and the efficient detection of low-copy number nucleic acids (e.g., DNA and/or RNA) in a given sample, there are no really effective methods relative to the standard PCR. It would be advantageous to invent systems, compositions, and methods that overcome these deficiencies. The present invention addresses these and related needs.

### BRIEF SUMMARY

The present technology relates generally to the field of genetic identification and detection of low-copy number nucleic acids, and provides method, compositions, kits, and systems useful for this purpose.

The invention describes methods, compositions, and systems for detecting microbial DNA or RNA, cfDNA and ctDNA, and genotyping or sequencing of low-copy number DNA, which can increase sensitivity of detection by more than ten-fold relative to conventional PCR methods, and/or yield purified targeted DNA, without of use of specific probes, suitable for use in Sanger sequencing or NGS (next generation sequencing) instruments. The methods of the invention not only provide over 10-fold higher sensitivity vs. PCR; they also advantageously reduce the number of steps and time during workflow DNA/RNA library preparation (for various genetic engineering purposes).

According to one aspect of the invention, a method is provided for identifying a low-copy number of a nucleic acid in a sample. The method comprises: providing a sample suspected of comprising a low-copy number of a nucleic acid; amplifying said nucleic acid to produce a first amplification products using two or more oligonucleotide primer pairs specific for said nucleic acid, wherein one or more of said oligonucleotide primers comprises one or more labels; generating a first amplification product that comprises sequences of said one or more labeled oligonucleotide primers; and detecting the presence of said amplification product using the one or more labeled oligonucleotide primers; wherein the presence of said amplification product detects a nucleic acid with a low-copy number. The method may further comprise the step of sequencing of the amplification product. Preferably, the nucleic acid is DNA or RNA. More preferably, the nucleic acid is from a coronavirus. Most preferably, the nucleic acid is from SARS-COV-2.

According to another aspect of the invention, a method is provided for identifying the presence of a coronavirus in a sample. The method comprises: providing a sample suspected of comprising a coronavirus; amplifying a nucleic acid from said coronavirus to produce a first amplification products using two or more oligonucleotide primer pairs specific for said coronavirus, wherein one or more of said oligonucleotide primers comprises one or more labels; generating a first amplification product that comprises sequences of said one or more labeled oligonucleotide primers; and detecting the presence of said amplification product using the one or more labeled oligonucleotide primers; wherein the presence of said amplification product detects the presence of the coronavirus in the sample. Preferably, the coronavirus is SARS-COV-2. The method may further comprise the step of sequencing of the amplification product.

According to another aspect of the invention, a system is provided for the identification of a low-copy number of a nucleic acid in a sample. The system comprises: a sample suspected of comprising a low-copy number of a nucleic acid, and two or more oligonucleotide primer pairs specific for said nucleic acid, wherein one or more of said oligonucleotide primers comprises one or more labels, wherein said one or more labeled oligonucleotide primer pairs are used to generate a first amplification product that comprises sequences of said one or more labeled oligonucleotide primer pairs, and wherein the one or more labeled oligonucleotide primers are used to detect said amplification product, thereby identifying a nucleic acid with a low-copy number. The amplification product can also be sequenced. Preferably, the nucleic acid is DNA or RNA. More preferably, the nucleic acid is from a coronavirus. Most preferably, the nucleic acid is from SARS-COV-2.

The foregoing is a summary and thus by necessity contains simplifications, generalizations and omissions of detail. Consequently, those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 depicts a description of one embodiment of the method, and a workflow for detection of microorganisms such as the SARS-COV-2.

FIG. 2 depicts the preparation of target DNA library for direct next generation sequencing (NGS).

FIG. 3 depicts the steps of a modified Sanger sequencing protocol for low-copy DNA samples.

FIG. 4 depicts data showing that the signal strength obtained using the methods of the instant invention is more than 10-fold increased relative to PCR.

FIG. 5 depicts results showing superior sensitivity of the instant detection methods.

FIG. 6 depicts results showing superior sensitivity of the instant detection methods for detection of low-copy number nucleic acids.

FIG. 7 depicts the detection “tune-up” feature of the instantly described methods, for the detection of extremely low quantities of DNA by increasing the volume of the PCR product load to the capillary electrophoresis instrument.

FIG. 8 depicts the use of the instant methods for manufacturing high quality affordable assays.

### SEQUENCE LISTING

The nucleic and amino acid sequences listed in the accompanying sequence listing are shown using standard letter abbreviations for nucleotide bases, and three letter code for amino acids, as defined in 37 C.F.R. § 1.822. Only one strand of each nucleic acid sequence is shown, but the complementary strand is understood as included by any reference to the displayed strand. The Sequence Listing is submitted as an ASCII text file, and is incorporated by reference herein.

SEQ ID NO: 1 is the nucleotide sequence of a nCoV_IP2 forward primer.

SEQ ID NO: 2 is the nucleotide sequence of a nCoV_IP2 reverse primer.

SEQ ID NO: 3 is the nucleotide sequence of a nCoV_IP4 forward primer.

SEQ ID NO: 4 is the nucleotide sequence of a nCoV_IP4 reverse primer.

SEQ ID NO: 5 is the nucleotide sequence of a nCoV_IP2 forward primer.

SEQ ID NO: 6 is the nucleotide sequence of a nCoV_IP2 reverse primer.

SEQ ID NO: 7 is the nucleotide sequence of a nCoV_IP4 forward primer.

SEQ ID NO: 8 is the nucleotide sequence of a nCoV_IP4 reverse primer.

SEQ ID NO: 9 is the nucleotide sequence of a nCoV_IP2 forward primer.

SEQ ID NO: 10 is the nucleotide sequence of a nCoV_IP2 reverse primer.

SEQ ID NO: 11 is the nucleotide sequence of a nCoV_IP4 forward primer.

SEQ ID NO: 12 is the nucleotide sequence of a nCoV_IP4 reverse primer.

### DETAILED DESCRIPTION OF THE PRESENTLY PREFERRED EMBODIMENTS

In the following detailed description, numerous specific details are set forth to provide a thorough understanding of the concepts underlying the described embodiments. It will be apparent, however, to one skilled in the art that the described embodiments can be practiced without some or all of these specific details. In other instances, well known process steps have not been described in detail in order to avoid unnecessarily obscuring the underlying concepts.

Broadly speaking the embodiments herein describe a new approach, which relates to compositions and methods for to achieving a major improvement in sensitivity of detection of low-copy DNA or RNA, and/or for enriching or selecting for targeted DNA without of loss of specificity and sensitivity due to use of probes in real-time RTPCR or in library preparation methods. Accordingly, a comprehensive solution to the issue of DNA and/or RNA detection was invented with the features described below.

As used herein, the words “a” and “an” mean “one or more.”

In one embodiment of the invention, the instant method of detection incorporates sensitivity as its primary goal.

In another embodiment of the invention, the method design assumes that the PCR primers are the criteria for specificity of the assay.

In another embodiment of the invention, the methods use an improved and/or modified PCR protocol, wherein one or both of the PCR primers used in the reaction are labeled with one or more detectable labels. The labeled primer(s) can be detected using methods that would be used by one of ordinary skill in the art. For example, following rounds of amplification of a suspected target nucleic acid, the detection of the labeled primers in the final product (e.g., amplicon) can serve to identify the presence of the target nucleic acid in the sample. In some preferred embodiments, one primer (e.g., forward or reverse) is labeled. In other preferred embodiments, two primers (e.g., both forward and reverse) are labeled. When more than one primer is labeled, the labels may be identical, or they may be different.

In another embodiment of the invention, the method design is reflective of the fact that the post-PCR mix consists of leftover fluorescently labeled and unlabeled primers, polymerase, buffer, dNTPs, genomic DNA, and other compounds and that the PCR product is in double-stranded configuration.

In another embodiment of the invention, the method design is reflective of the fact that most of currently available instrument-platforms designed to detect the DNA or RNA material are usually detecting labels, like fluorescent dyes, radioactive labels, or other tags usually attached to the targeted DNA or RNA.

In another embodiment of the invention, the method assumes that these DNA-labels are used by the instrument for detection of the target DNA.

In another embodiment of the invention, the method assumes that the targeted-DNA can be detected either by capillary-based, or real-time RT-PCR instruments.

In another embodiment of the invention, the method design is reflective of the fact that the sensitivity of the most instruments or platforms for detection or interrogation of the post-PCR products is impeded by so called “background noise” that is usually part of the PCR product such as, unincorporated primers, dNTPs, enzyme, buffer, ions, genomic DNA, etc.

In another embodiment of the invention, the method uses at least one of the PCR primers as a probe for the detection of the target sequence, therefore, excluding the need of separate probe(s) usually used with a real-time RT-PCR assays, or selecting the targeted sequences by hybridizing to specific probes during library preparation methods for Sangers and NGS sequencing.

In another embodiment of the invention, the PCR mix consists of primers, polymerase, buffer, dNTPs, etc., as understood by one of ordinary skill in the art. The forward primer is dye-labeled at a 5′-end and the reverse primer is labeled with biotin at the 5′-end. After the PCR, the intended amplification target is in double-stranded configuration, dye-labeled on one strand, and biotin-labeled on the other.

In another embodiment of the invention, after the PCR amplification, biotin-labeled double-stranded product is captured to the streptavidin coated surface.

In another embodiment of the invention, washing of unincorporated dye-labeled and unlabeled primers and other artifacts follows the capturing phase.

In another embodiment of the invention, after the washing, the targeted labeled DNA product is released by denaturation. This yields a highly concentrated dye-labeled, single-stranded DNA or RNA target, that is ready to be loaded to an instrument for genotyping on capillary electrophoresis platform.

In another embodiment of the invention, if a real-time RT-PCR instrument is used for detection of the targeted DNA or RNA, the role of probe is assumed by the PCR primers, and there will be no threshold line and background noise will be minimal.

In another embodiment of the invention, the PCR primers can be specific to the follow up detection of the target regarding the final goal. If the goal is to obtain a genotype for an identification purposes of a microorganism or marker for cancer, then the primers would not need additional sequences (nucleotides). However, if the goal is to obtain sequence of the targeted DNA, then the PCR primers have to incorporate at the 5′-ends the so-called adapter sequences that are complementary to the capturing sequences that are integral part of and are used by the sequencing platforms.

The invention is particularly useful for the detection in a given sample the presence of nucleic acids (e.g., DNA and/or RNA) of various microorganisms, including SARS-COV-2.

In yet another embodiment of the invention, at least one of the primers is labeled using methods and compositions known in the art. Exemplary labels include radioactive markers, fluorescent markers, digoxigenin, and others.

The labeled primer(s) hereof may be used with any nucleic acid target. These target sequences may include, but are not limited to, DNA, RNA, chromosomal or purified nuclear DNA, heteronuclear RNA, and other nucleic acids.

“Primer” means a short strand of oligonucleotides complementary to a specific target sequence of DNA which is used to prime DNA synthesis. The primers of the invention preferably should have a length of at least about5, 10, 15, 20, 25, 30, 35, 40, 45, 50, 55, 60, 65, 70, 75, 80, 85, 90, 95, or 100 nucleotides. Labeled primers of this length are generally sufficient for the identification or detection of the target nucleic acid.

“Hybridization” refers to the process of joining two complementary strands of nucleic acid (e.g., DNA) to form a double-stranded molecule.

“Nucleotide” means a building block of DNA or RNA, consisting of one nitrogenous base, one phosphate molecule, and one sugar molecule (deoxyribose in DNA, ribose in RNA).

“Oligonucleotide” means a short string of nucleotides. Oligonucleotides are often used as primers and/or probes, to find a matching sequence of DNA or RNA. Oligonucleotides can be labeled with a variety of labels, such as radioisotopes and fluorescent and chemiluminescent moieties.

The term “system for identifying” or “detection system” as used herein refers to a method that enables visualization of amplified nucleic acid products. Examples of suitable systems for identification (detection systems) include systems that utilize labels. The identification or detection may depend on radioactive exposure, fluorescence and chemiluminescence, using methods and compositions that are known in the art.

Amplification of nucleic acids is accomplished using methods and compositions known in the art, for example polymerase chain reaction (PCR).

The term “amplicon” as used herein, refers to a segment of a polynucleotide which is amplified in an amplification reaction. The instant invention is particularly useful for the generation and detection of amplicons that result from the amplification of low-copy number nucleic acids.

The invention further contemplates equivalents to the systems, compositions, and methods described herein. Therefore, the invention is not restricted to the preferred embodiments described and illustrated but covers all modifications and equivalents. In the ensuing detailed explanation, the usual case of a DNA target sequence and DNA primers is discussed; however, those skilled in the art will understand that the discussion is equally applicable (with art-recognized differences owing to the nature of the target sequences and probes) to other nucleic acid species.

### EXEMPLARY PREFERRED EMBODIMENTS

Embodiment 1: a method for identifying a low-copy number of a nucleic acid in a sample, the method comprising: providing a sample suspected of comprising a low-copy number of a nucleic acid; amplifying said nucleic acid to produce a first amplification products using two or more oligonucleotide primer pairs specific for said nucleic acid, wherein one or more of said oligonucleotide primers comprises one or more labels; generating a first amplification product that comprises sequences of said one or more labeled oligonucleotide primers; and detecting the presence of said amplification product using the one or more labeled oligonucleotide primers, wherein the presence of said amplification product detects a nucleic acid with a low-copy number.

Embodiment 2: the embodiment 1 (above), wherein the amplification product is sequenced.

Embodiment 3: the embodiment 1 (above), wherein the nucleic acid is DNA or RNA.

Embodiment 4: the embodiment 1 (above), wherein the nucleic acid is from a coronavirus.

Embodiment 5: the embodiment 4 (above), wherein the nucleic acid is from SARS-COV-2.

Embodiment 6: the embodiment 1 (above), wherein the nucleic acid is amplified with a polymerase chain reaction (PCR).

Embodiment 7: a method for identifying the presence of a coronavirus in a sample, the method comprising: providing a sample suspected of comprising a coronavirus, amplifying a nucleic acid from said coronavirus to produce a first amplification products using two or more oligonucleotide primer pairs specific for said coronavirus, wherein one or more of said oligonucleotide primers comprises one or more labels, generating a first amplification product that comprises sequences of said one or more labeled oligonucleotide primers, and detecting the presence of said amplification product using the one or more labeled oligonucleotide primers, wherein the presence of said amplification product detects the presence of the coronavirus in the sample.

Embodiment 8: the embodiment 7 (above), wherein the amplification product is sequenced.

Embodiment 9: the embodiment 7 (above) wherein the nucleic acid is DNA or RNA

Embodiment 10: the embodiment 7 (above), wherein the coronavirus is SARS-COV-2.

Embodiment 11: the embodiment 7 (above), wherein the nucleic acid is amplified with a polymerase chain reaction (PCR).

Embodiment 12: a system for identifying a low-copy number of a nucleic acid in a sample, the system comprising: a sample suspected of comprising a nucleic acid, and one or more oligonucleotide primer pairs specific for said nucleic acid, wherein one or more of said oligonucleotide primers from said primer pairs comprises one or more labels, wherein said one or more labeled oligonucleotide primer pairs are used to generate a first amplification product that comprises sequences of said one or more labeled oligonucleotide primers, and wherein the one or more labeled oligonucleotide primers are used to detect said amplification product, thereby identifying a nucleic acid with a low-copy number.

Embodiment 13: the embodiment 12 (above), wherein the nucleic acid is a low-copy number nucleic acid.

Embodiment 14: the embodiment 12 (above), wherein the amplification product is sequenced.

Embodiment 15: the embodiment 12 (above), wherein the nucleic acid is DNA or RNA.

Embodiment 16: the embodiment 12 (above), wherein the nucleic acid is from a coronavirus.

Embodiment 17: the embodiment 16 (above), wherein the nucleic acid is from SARS-COV-2.

Embodiment 18: the embodiment 12 (above), wherein the nucleic acid is amplified with a polymerase chain reaction (PCR).

### EXAMPLES

### Example 1

Description of the Method and Workflow for Detection of Microorganisms Such as SARS-COV-2 (COVID-19).

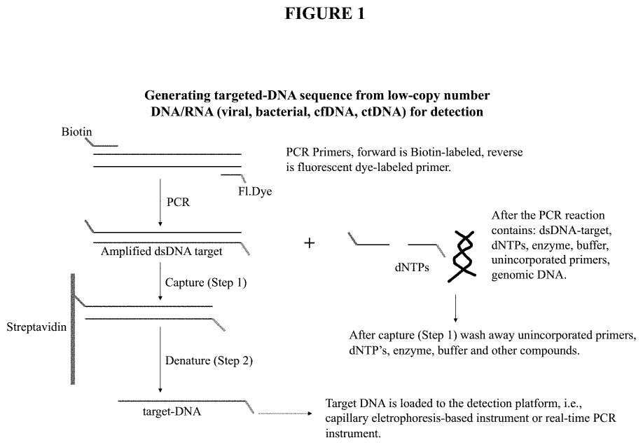

The following is a description of an exemplary method for detection of SARS-COV-2. See also FIG. 1.
1. Extraction of viral RNA.
2. Reverse Transcriptase PCR to transform viral RNA to cDNA.
3. PCR-amplification of the cDNA by SARS-COV-2 primers such as nCoV_IP2 and nCoV_IP4 primer pairs, for example.

In this example, PCR amplification of the cDNA with specific primers where 5′-end of the forward primer is labeled with Biotin and the 5′-end of the reverse primer is labeled with fluorescent dye. Of course, the use of other detectable labels is equally possible.

Primer set nCoV_IP2 expected product size 108 bp.

Forward:
(SEQ ID NO: 1)
5′-Biotin-ATGAGCTTAGTCCTGTTG-3′
Reverse:
(SEQ ID NO: 2)
5′-HEX-CTCCCTTTGTTGTGTTGT-3′

Primer set nCoV_IP4 expected product size 107 bp.

Forward:
(SEQ ID NO: 3)
5′-Biotin-GGTAACTGGTATGATTTCG-3′
Reverse:
(SEQ ID NO: 4)
5′-FAM-CTGGTCAAGGTTAATATAGG-3′

Another primer pair for detection of SARS-COV-2 could be:

Forward:
(SEQ ID NO: 5)
5′-Biotin-GACCCCAAAATCAGCGAAAT-3′
Reverse:
(SEQ ID NO: 6)
5′-FAM-TCTGGTTACTGCCAGTTGAATCTG-3′

Another primer pair for detection of SARS-COV-2 could be:

Forward:
(SEQ ID NO: 7)
5′-Biotin-TTACAAACATTGGCCGCAAA-3′
Reverse:
(SEQ ID NO: 8)
5′-FAM-GCGCGACATTCCGAAGAA-3′

Another primer pair for detection of SARS-COV-2 could be:

Forward:
(SEQ ID NO: 9)
5′-Biotin-AGATTTGGACCTGCGAGCG-3′
Reverse:
(SEQ ID NO: 10)
5′-FAM-GAGCGGCTGTCTCCACAAGT-3′

Another primer pair for detection of SARS-COV-2 could be:

Forward:
(SEQ ID NO: 11)
5′-Biotin-GGGAGCCTTGAATACACCAAAA-3′
Reverse:
(SEQ ID NO: 12)
5′-FAM-TGTAGCACGATTGCAGCATTG-3′

In some embodiments of the invention, one or more of the primers above, or other specific primers, can be used for obtaining the primary structure of the targeted DNA or RNA (such as SARS-COV-2) as a part of sequencing reaction (NGS- or -Sanger-sequencing). For example, a 5 μl of extracted and eluted RNA can be added to combined reverse-transcriptase-PCR-amplification reaction for a total of 25 μl that can contain the following amplification mix:

| Simplex Mix Vol (μl) | [final] | | |
|---|---|---|---|
| H₂O PPI | 3.60 | | |
| Reaction mix 2X | 13.00 | 3.0 | mM Mg |
| MgSO4 (50 mM) | 0.40 | 0.8 | mM Mg |
| Forward Primer (10 μM) | 1.00 | 0.4 | μM |
| Reverse Primer (10 μM) | 1.00 | 0.4 | μM |
| Reverse Transc./Taq Pol enz. Mix | 1.00 | | |
| Final Volume | 20.00 | | |

An example of a cycle protocol is as follows:
Reverse transcription 55° C. 20 min xl
Denaturation 95° C. 3 min xl
Amplification 95° C. 15 sec followed by 58° C. 30 sec×50
Cooling 40° C. 30 sec xl
4. Schematic diagram of the PCR starting with the viral cDNA is shown in FIG. 1.
5. The PCR product after the washing step consists of 100% intended target ssDNA that is dye-labeled and in configuration that maximizes detection limits of the capillary electrophoresis or real-time RT-PCR platform.
6. The amplified target-DNA is loaded to a detection platform, that can be either capillary electrophoresis or real-time RT-PCR instrument.
7. Positive control can be generated by amplification of synthetic SARS-CO 2 DNA.
8. Negative control can be generated with no-template DNA in the PCR Mix followed by the above the workflow.

### Example 2

Target DNA Library Preparation for Direct NGS

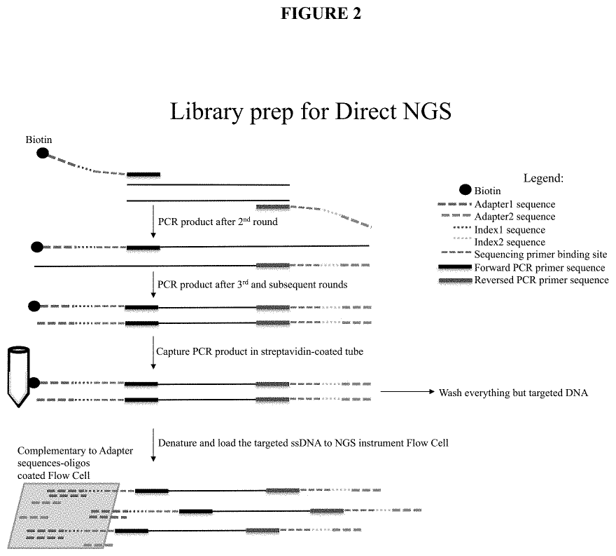

If the goal is to produce a targeted ssDNA for Sangers or NGS experiment, the PCR primers will be outflanked by adapter sequences, complementary to the oligos attached on the surface of the flow cell used by NGS platform. In one PCR-reaction the forward primer will be Biotin-labeled but the reverse (the targeted strand) unlabeled. In the second PCR the forward primer (targeted strand) will be unlabeled but the reverse will be Biotin-labeled. This way, both, the forward and reverse strands of the DNA can be produced in separate PCR reactions, combined in equimolar volumes before capturing, and following the washing step, can be loaded to the flow cell that is part of the NGS instrument. However, this may not be necessary if the instrument can use only DNA in either forward or reverse single strand DNA configuration. For example, Illumina NGS technology requires loading of ssDNA targets in both, forward and reverse configuration, that are captured by complementary-to-the-adapter sequences oligos attached to the surface of the Flow Cell, and after a Cluster Generation process, strands in reversed configuration are cleaved and washed away and forward strands are sequenced which is followed by round of Cluster Generation after which forward strands are cleaved and washed away followed by sequencing of the reverse strands. However, if the DNA target that is loaded to the Flow Cell is in only one orientation (e.g., forward orientation) there is no necessity to have a cleavage and washing step before the first round of sequencing begins. This may save time and reagents during NGS but would require reprograming of the NGS-workflow controlling software. The workflow of this process is shown in FIG. 2.

Library Prep workflow for direct NGS sequencing (above) would typically take no longer than 2 hours and consists of only 4 steps:

PCR: amplification of the targeted DNA by overhang PCR primers that would add the adapter, index and sequence primer-binding site to the primers complementary to the targeted sequence. After the first two rounds of the PCR, the overhang sequences become a component of the PCR product.

Capturing: Isolation of the Biotin-labeled targeted DNA that is in double-strand configuration (dsDNA) to the surface of the streptavidin-coated loading tube. Here, the Streptavidin coating is quantified to capture only desired number of copies of dsDNA target, and this would be the quantity of the DNA-load that is optimal for the particular NGS instrument, thus, eliminating time-consuming and costly DNA-load quantification step. In addition, due to PCR-primer specificity, described Library prep method, eliminates a need for qualitative analysis of the target (confirming the size of the target DNA), additionally saving time and resources during Library prep procedure.

Washing of all PCR leftovers but the attached to the tube wall the dsDNA target.

Releasing by denaturant the ssDNA target and transferring the loading tube to the reagent cartridge to be loaded directly to the Flow Cell of the NGS instrument.

Alternatively, the genomic DNA could be fragmented and tagged, this would produce a plurality of dsDNA products of equal sizes (about 300 bp) with ends complementary to the PCR primers, and the workflow will continue per FIG. 2.

### Example 3

Modified Sanger Sequencing Protocol for Low-Copy DNA Samples

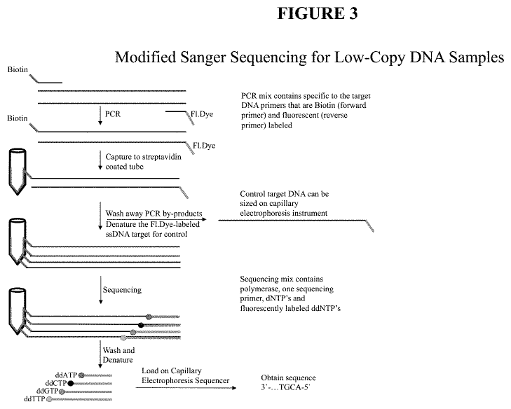

If the DNA samples consist of degraded or low-copy DNA such as the case with cfDNA and ctDNA samples the targeted DNA can be first amplified by PCR with one biotinylated primer followed by the capturing of the target product on streptavidin coated tubes, this will be followed by washing step, denaturing and releasing of ssDNA target that can be used as a control, then the sequencing reaction mix (containing dNTP's and fluorescently labeled ddNTPs), can be included in the same tube where the target DNA is in single stranded DNA configuration that is bind to the reaction tube's wall, and following the one or more cycles of sequencing (linear extension), after a washing step, the ssDNA products with terminal, fluorescently-labeled ddNTPs 3′-ends of various sizes can be denatured and loaded on the capillary electrophoresis instrument that will read the DNA sequence (see FIG. 3).

### Example 4

Signal Strength Obtained Using the Methods of the Instant Invention is More than 10-Fold Increased Relative to PCR

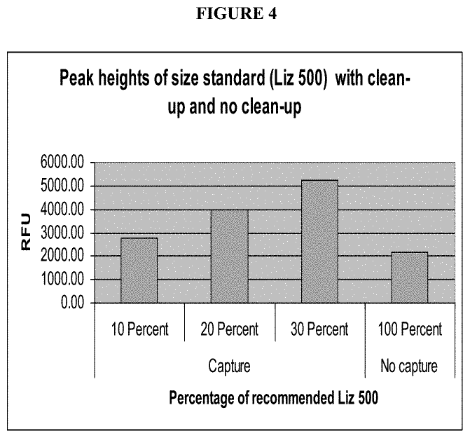

Sensitivity of a PCR-assay is the key factor to an early viral detection. For example, it is generally understood that the PCR-based assays are about 100× more sensitive than ELISA-based assays. The PCR workflow described above is typically at least 10 times more sensitive than the currently used PCR assays for detection of viral DNA (such as SARS-COV-2). For example, the signal strength obtained using the methods of the instant invention is more than 10-fold increased relative to classical PCR workflow; see FIG. 4. As shown in FIG. 4, 10% of Liz 500 size standard recommended volume produced by the modified PCR workflow described above, generated higher signal strength than 100% of recommended LIZ500 volume generated by the “classical” PCR.

### Example 5

Superior Sensitivity of the Instant Detection Methods

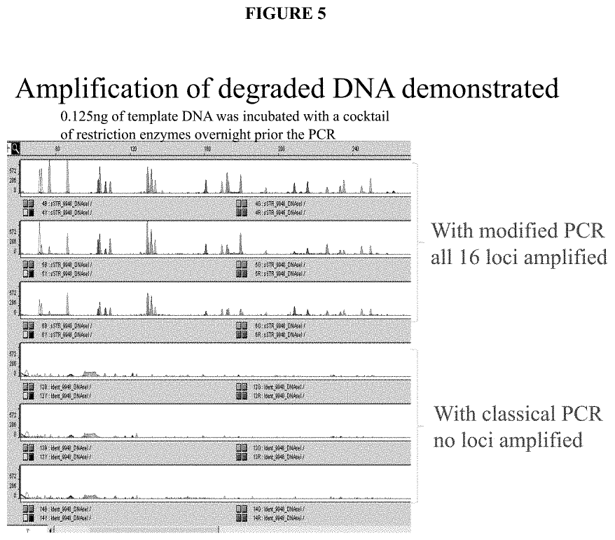

Sensitivity of the modified PCR protocol of the instant invention was demonstrated in the experiments summarized in FIG. 5. When DNA fully degraded by restriction enzyme was amplified by 16-Plex PCR with modified protocol, all loci were visible (top three electropherograms). In contrast, no loci were visible after PCR amplification (lower three electropherograms) of the same degraded DNA after running the sample on the capillary electrophoresis instrument, shown in FIG. 5.

### Example 6

Superior Sensitivity of the Instant Detection Methods for Detection of Low-Copy Number Nucleic Acids

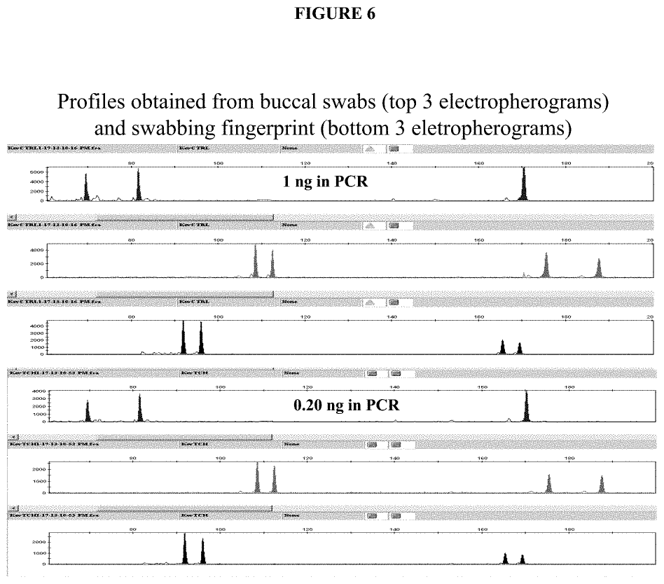

Additional sensitivity in amplifying a low-copy DNA with the instantly described methods was demonstrated during the comparison of 6-Plex PCR amplification of DNA extracted from cheek buccal swabs vs DNA collected by swabbing a fingerprint on a metal surface (FIG. 6). Genotype based on six amplified loci could reliably be obtained from the fingerprint swabs (lower three electropherograms) when PCR product was separated by capillary electrophoresis.

### Example 7

“Tune Up” of the Detection Method

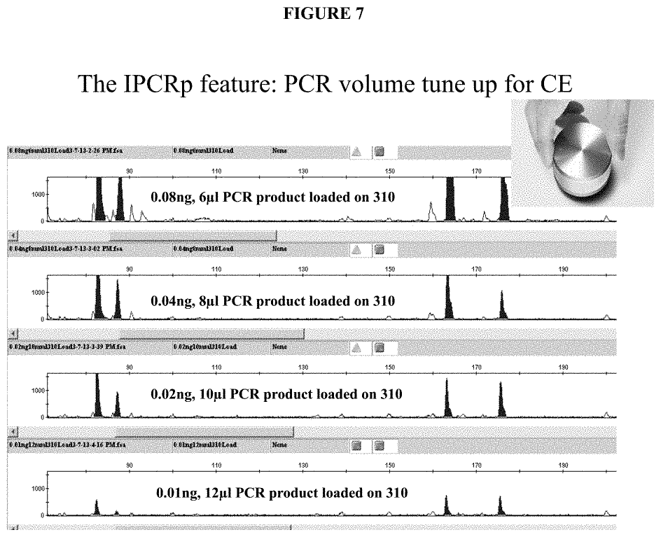

The detection “tune-up” feature of the instantly described methods, similar to the volume tune-up button on the stereo instrument, was demonstrated when the extremely low quantities of DNA were detected by increasing the volume of the PCR product load to the capillary electrophoresis instrument (FIG. 7). Increasing of background noise is associated with higher load quantities of regular PCR, therefore, excluding as an option for sensitivity increase.

### Example 8

Manufacturing of High-Quality Affordable Assays

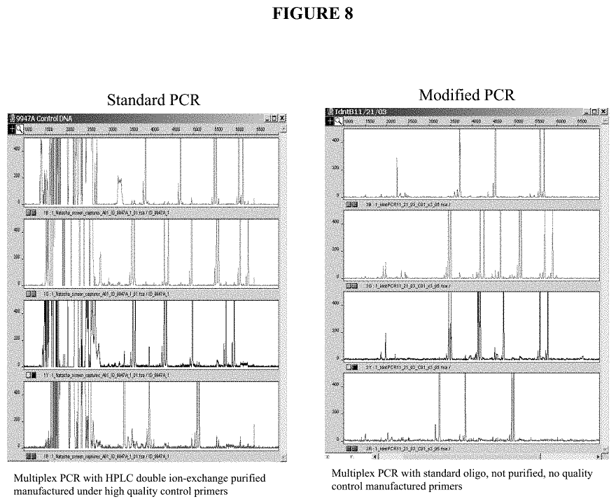

Manufacturing of high quality oligos can be expensive and time-consuming process that can be a limiting factor in designing affordable PCR-assays. However, quality of the data produced by the modified PCR method described herein is not affected by the quality of the oligonucleotides. The background noise of the regular PCR conducted by expensive high-quality primers is much higher than the background noise of the modified PCR conducted by inexpensive and easy-to-manufacture regular quality primers (FIG. 8).

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters normally encountered in the fields of fungicides and treatments of plants and soil, obvious to those skilled in the art, are within the scope of this invention. All publications, patents, and patent applications cited herein are incorporated by reference in their entirety for all purposes.

What is claimed is:

1. A method for identifying a low-copy number of less than 1 ng of a genomic nucleic acid in a sample, the method comprising:
a. providing a sample of comprising a low-copy number of less than 1 ng of a genomic nucleic acid;
b. amplifying said nucleic acid using Polymerase Chain Reaction (PCR) to produce a first amplification products using two or more oligonucleotide primers specific for said nucleic acid, wherein one or more of said oligonucleotide primers comprises one or more labels;
c. generating a first amplification product that comprises sequences of said one or more labeled oligonucleotide primers; and
d. detecting the presence of said amplification product using the one or more labeled oligonucleotide primers;
e. wherein the presence of said amplification product detects a nucleic acid with a low-copy number in the amount of less than 1 ng; and
f. wherein the signal strength of said amplified product is more than 10-fold increased relative to a comparable PCR workflow which uses two or more oligonucleotide primers specific for said genomic nucleic acid, and wherein said one or more oligonucleotide primers do not comprise one or more labels.

2. The method of claim 1, further comprising the step of sequencing of the amplification product.

3. The method of claim 1, wherein the nucleic acid is DNA or RNA.

4. The method of claim 1, wherein the nucleic acid is from a coronavirus.

5. The method of claim 4, wherein the nucleic acid is from SARS-COV-2.

6. (canceled)

7. A method for identifying the presence of a low-copy number of coronavirus nucleic acid in a sample, the method comprising:
a. providing a sample comprising less than 1 ng of a coronavirus nucleic acid;
b. amplifying the nucleic acid from said coronavirus using PCR to produce a first amplification product using two or more oligonucleotide primers specific for said coronavirus nucleic acid, wherein one or more of said oligonucleotide primers comprises one or more labels;
c. generating a first amplification product that comprises sequences of said one or more labeled oligonucleotide primers; and
d. detecting the presence of said amplification product using the one or more labeled oligonucleotide primers;
e. wherein the signal strength of said amplified product is more than 10-fold increased relative to a comparable PCR workflow which uses two or more oligonucleotide primers specific for said coronavirus nucleic acid, and wherein said one or more oligonucleotide primers do not comprise one or more labels;
f. wherein the presence of said amplification product detects less than 1 ng of said nucleic acid from said coronavirus;
g. thereby detecting the presence of less than 1 ng of the coronavirus nucleic acid in the sample.

8. The method of claim 7, further comprising the step of sequencing of the amplification product.

9. The method of claim 7, wherein the nucleic acid is DNA or RNA.

10. The method of claim 7, wherein the coronavirus is SARS-COV-2.

11. (canceled)

12. A system for identifying less than 1 ng of a genomic nucleic acid in a sample, the system comprising:
a. a sample comprising a low-copy number of less than 1 ng of a genomic nucleic acid; and
b. two or more oligonucleotide primers specific for said genomic nucleic acid, wherein one or more of said oligonucleotide primers comprises one or more labels;
c. wherein said one or more labeled oligonucleotide primers are used in a PCR reaction to generate a first amplification product that comprises sequences of said one or more labeled oligonucleotide primer pairs; and
d. wherein the one or more labeled oligonucleotide primers are used to detect said amplification product;
e. thereby identifying the genomic nucleic acid;
f. wherein the signal strength of said amplified product is more than 10-fold increased relative to a comparable PCR workflow which uses two or more oligonucleotide primers specific for said genomic nucleic acid, and wherein said one or more oligonucleotide primers do not comprise one or more labels.

13. The system of claim 12, wherein the amplification product is sequenced.

14. (canceled)

15. (canceled)

16. The system of claim 12, wherein the nucleic acid is from a coronavirus.

17. The system of claim 16, wherein the nucleic acid is from SARS-COV-2.

18. (canceled)


SEQUENCE LISTING (12 sequences)

SEQ ID NO: 1 - DNA, 18 bp, Artificial sequence
  nCoV_IP2 forward primer
atgagcttagtcctgttg

SEQ ID NO: 2 - DNA, 18 bp, Artificial sequence
  nCoV_IP2 reverse primer
ctccctttgttgtgttgt

SEQ ID NO: 3 - DNA, 19 bp, Artificial sequence
  nCoV_IP4 forward primer
ggtaactggtatgatttcg

SEQ ID NO: 4 - DNA, 20 bp, Artificial sequence
  nCoV_IP4 reverse primer
ctggtcaaggttaatatagg

SEQ ID NO: 5 - DNA, 20 bp, Artificial sequence
  Forward primer for SARS-COV-2
gaccccaaaatcagcgaaat

SEQ ID NO: 6 - DNA, 24 bp, Artificial sequence
  Reverse primer for SARS-COV-2
tctggttactgccagttgaatctg

SEQ ID NO: 7 - DNA, 20 bp, Artificial sequence
  Forward primer for SARS-COV-2
ttacaaacattggccgcaaa

SEQ ID NO: 8 - DNA, 18 bp, Artificial sequence
  Reverse primer for SARS-COV-2
gcgcgacattccgaagaa

SEQ ID NO: 9 - DNA, 19 bp, Artificial sequence
  Forward primer for SARS-COV-2
agatttggacctgcgagcg

SEQ ID NO: 10 - DNA, 20 bp, Artificial sequence
  Reverse primer for SARS-COV-2
gagcggctgtctccacaagt

SEQ ID NO: 11 - DNA, 22 bp, Artificial sequence
  Forward primer for SARS-COV-2
gggagccttgaatacaccaaaa

SEQ ID NO: 12 - DNA, 21 bp, Artificial sequence
  Reverse primer for SARS-COV-2
tgtagcacgattgcagcattg